(19) United States
(12) Patent Application Publication
(10) Pub. No.: US 2017/0086886 A1
(43) Pub. Date: Mar. 30, 2017

(54) MODULAR BONE SCREW ASSEMBLY

(71) Applicant: Amendia, Inc., Marietta, GA (US)

(72) Inventors: Jim Duncan, Hernando, MS (US); Daniel Sayger, Southaven, MS (US); Kenneth Richard Barra, Acworth, GA (US)

(73) Assignee: Amendia, Inc., Marietta, GA (US)

(21) Appl. No.: 14/871,125

(22) Filed: Sep. 30, 2015

(51) Int. Cl. A61B 17/70

(52) U.S. Cl. CPC A61B 17/7037; A61B 17/7035

(57) ABSTRACT

A modular head bone screw assembly has a bone screw, a tulip and a saddle. The saddle is positioned in a recess inside of the tulip. The saddle has a proximal end for engaging a rod and a distal end for receiving a hemispherical or at least partial hemispherical head of the bone screw. The saddle is sized to move axially inside the tulip. The saddle has a plurality of fingers separated by slots extending from near the proximal end through the distal end in an initial un-loaded position and upon insertion of the tulip over the head of the bone screw, the saddle moves proximally over the head simultaneously causing the fingers to move past a maximum diameter of the hemispherical head and upon tightening the rod by a set screw, the fingers locking the bone screw into the tulip.

### TECHNICAL FIELD

The present invention relates to an improved modular head bone screw assembly.

### BACKGROUND OF THE INVENTION

Bone anchor screws come in a variety of shapes and sizes. One of the more common styles has a polyaxial head that allows for the screw to enter the bone structure at an ideal or preferred inclination. To achieve this polyaxial inclination, the head has a shape configured to rotate about its lower external surface. This lower surface can be one of a number of shapes like conical or spherical or hemispherical. This ability is often used in devices having a modular head assembly.

The bone screw assembly generally includes a tulip. A tulip is a body structure having two opposing sides spaced by a slotted opening to receive a spinal rod. The tulip often employs internal threads to receive a rod locking set screw to anchor or fix the rod in the tulip. The lower portion of the tulip has an opening to receive the bone screw in a base seat. Often, the tulip can have a saddle that both supports the rod along an underside of the rod. The saddle having an upper recessed curvature into which the rod sits and a lower cup like opening to receive the top of the bone screw head. When the saddle and rod and set screw are tightened, the screw angle is fixed against the tulip seat.

Often, it is preferred that the bone screw is first placed securely in the bone structure leaving the head protruding above the bone surface. In this surgical procedure the tulip assembly must be adapted to fit down onto the projecting screw head. To accomplish this, the surgeon must push the tulip onto and over the screw head without a clear path of vision. Accordingly, the placement must be accomplished without any way of knowing if the tulip or other device is properly secured. Thereafter, the device is tightened to complete the assembly and the only way to insure the assembly is secure requires an upward pulling of the tightened assembly. This is not a good test the assembly will be loosened or the screw to bone interface weakened.

It is, therefore, an objective of the present invention to provide a way for a surgeon to place a tulip assembly onto a bone screw already threaded into bone in such a way the surgeon can make a proper and secure connection easily.

It is a further objective that the device has properly fitted the tulip onto the bone screw head by the very nature of the design. It is another objective that the device provides a self-locking feature that when tightened by assembly, the surgeon can lock the assembly engagement insuring he has made a proper assembly. These and other objectives are achieved by the invention as described hereinafter.

### SUMMARY OF THE INVENTION

A modular head bone screw assembly has a bone screw, a tulip and a saddle. The tulip has a tapered distal end and a slotted opening for receiving a rod defined by a pair of sides. Each side has a proximal end with threads for engaging a set screw to secure the rod. The annular tapered distal end has an opening for receiving and securing the bone screw. The sides have opposing concave arcuate slots positioned interposed between the thread and tapered distal end. The saddle is internal of the tulip positioned in a recess inside of the tulip. The saddle has a proximal end for engaging a rod and a distal end for receiving a hemispherical or at least partial hemispherical head of the bone screw. The saddle has an exterior or outer surface positioned between the ends. The saddle is sized to move axially inside the tulip aligned by a pair of complimentary convex arcuate projections. Each projection is configured to enter one of said opposing slots. The saddle has a plurality of arcuate fingers positioned to create a bulbous exterior shape with an interior receiving chamber. The arcuate fingers are separated by slots extending from near the proximal end through the distal end in an initial pre-loaded position. The plurality of arcuate fingers collectively are larger in diameter than the tapered end annular opening of the tulip. Upon insertion of the tulip over the head of the bone screw, the saddle moves proximally over the head simultaneously causing the arcuate fingers to flex and move past a maximum diameter of the head holding the head in the complimentary shaped interior receiving chamber and upon tightening the rod by the set screw, the plurality of arcuate fingers are compressed about the head of the bone screw by the distal tapered end thereby fixing the bone screw into the tulip. Each finger has an arcuate shape with an inwardly positioned chamfered end for sliding on the surface of the head of the bone screw thereby flexing the plurality of arcuate fingers.

The recess of the tulip can be a conical surface tapering inward distally. The conical surface compresses the plurality of arcuate fingers when tightening the set screw. The relaxed outer diameter of the bulbous exterior of the saddle is larger than a distal opening of the tulip wherein as a rod is fixed into the proximal end by tightening a set screw, the distal end of the saddle conforms compressing the fingers to engage the outer surface of the hemispherical head below the maximum diameter. The fingers are sized to flex inwardly or outwardly. The saddle has preferably 6 or more fingers separated by slots. The inner surface of the saddle along an inner surface of the plurality of fingers forms the complimentary head receiving chamber in a hemispherical or at least partial hemispherical shape sized to pass over and past the maximum diameter of the head of the bone screw. When the fingers are so positioned over the head, the screw is held, but free to move. When the set screw is tightened, the fingers are compressed, the screw is fixed and its angularity is set.

The bone screw has one of the following head shapes; at least partially a hemispherical or spherical head, or any other bulbous head. The head has a driving feature for torsionally driving the screw into bone.

A method of assembling a tulip comprises the step of providing a tulip. The method also includes the step of positioning a saddle inside the tulip in the expanded condition. The method also includes the step of inserting a rod into the saddle and tightening the rod by a set screw threaded into the tulip thereby moving the saddle distally to engage and fix the head of the screw below the maximum diameter.

### BRIEF DESCRIPTION OF THE DRAWINGS

The invention will be described by way of example and with reference to the accompanying drawings in which:

FIG. 1 is a plan view of a tulip of the present invention.

FIG. 2 is a cross-sectional view of a tulip and saddle assembly above a head portion of a bone screw.

FIG. 3 is a perspective view of the saddle of the present invention.

FIG. 4 is a cross-sectional view of the tulip and saddle assembly shown positioned onto the head of a bone screw in a loose or untightened condition.

FIG. 5 is a view showing the saddle moved to the tightened positioned.

FIG. 6 is a cross-sectional assembly plan view of the tulip, saddle assembly tightened about the head of a bone screw showing the rod held in the saddle and fixed by a set screw; the rod and set screw when so assembled, drive the saddle distally compressing the fingers.

FIG. 7 is a perspective view of the view of FIG. 6.

FIG. 8 is a view of an exemplary pedicle or bone screw used with the present invention.

FIG. 9 is a view of the rod used with the present invention.

FIG. 10 is a view of the set screw used with the present invention.

FIG. 11 shows how a release tool engages the saddle of the present invention.

FIG. 12 shows a plan view of the release tool.

### DETAILED DESCRIPTION OF THE INVENTION

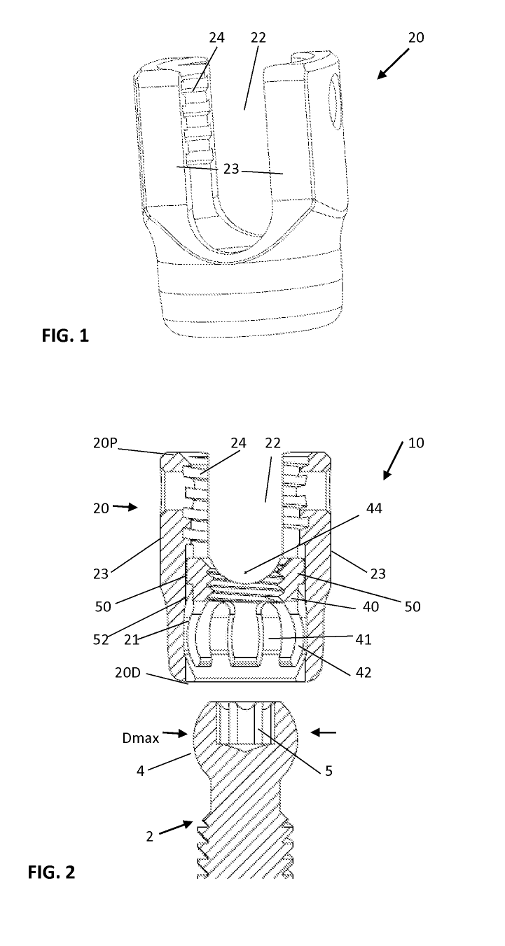

With reference to FIGS. 1-5, a first embodiment of the present invention is illustrated. In FIG. 1, a tulip 20 is shown in a plan view. The tulip 20 has a slotted opening 22 defined by opposed sides 23 to receive a rod and has internal threads 24 into which a set screw can be inserted that allow the rod to be positioned into the tulip 20 on assembly.

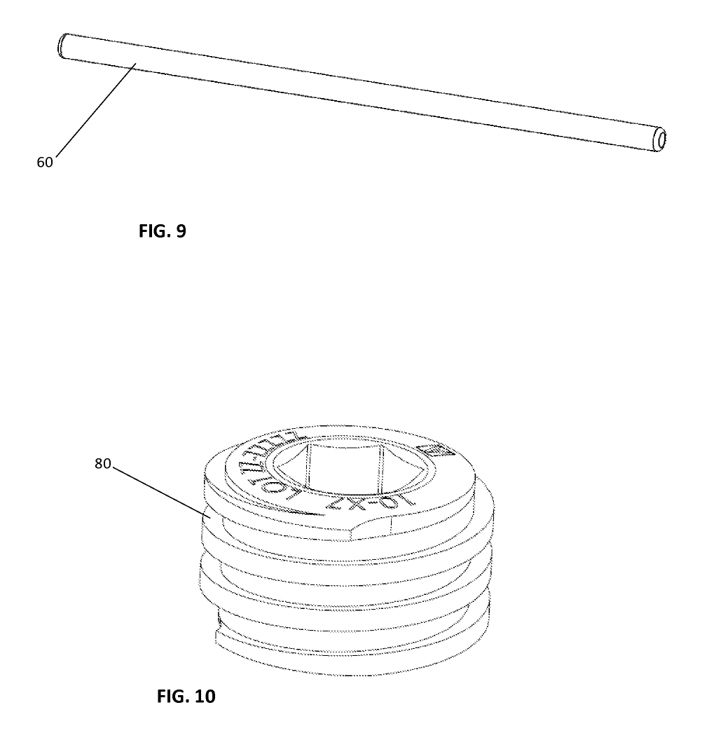

With reference to FIG. 2, a cross-sectional view of the assembly 10 is illustrated, internal of the tulip 20 is a recess. The proximal end 20P is shown below that internal of the tulip are the threads 24 for receiving the set screw illustrated later in FIGS. 9 and 10. Below the threads 24 is a recess portion 21 the tulip 20 with a saddle 40 illustrated inside the recess 21. As shown, the saddle 40 also has a concavity 44 for receiving a rod, this concavity 44 is in the form of a partially circular arc configured to the shape of the rod which it will receive and hold tightly upon final assembly. The saddle 40 has a proximal end for rod receiving and a distal end for receiving the head 4 of bone screw 2. The bone screw 2 is partially shown below the assembly of the tulip 20 and the saddle 40.

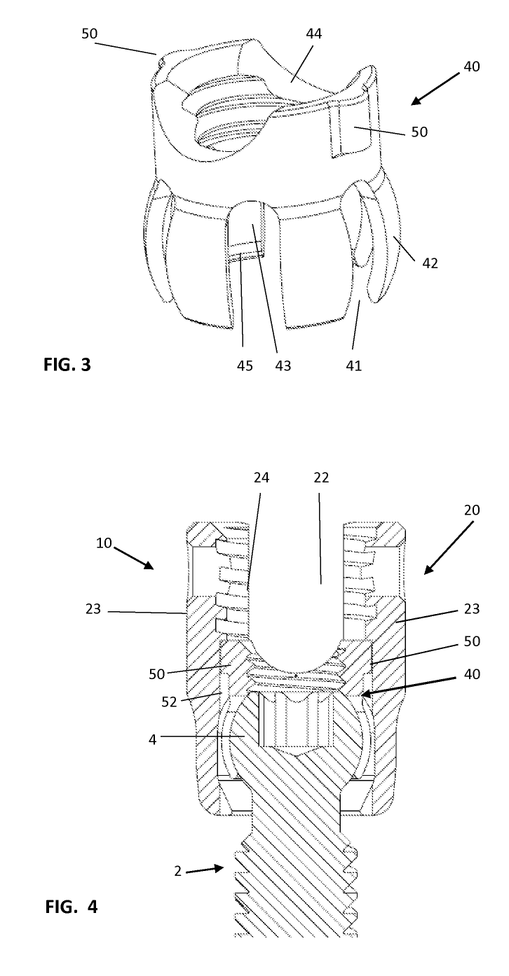

As further illustrated in FIG. 3, with reference to the saddle 40, a plurality of fingers 42 are shown defined by the slots 41 cut in near proximity to the recess 44 for receiving the rod near the proximal end and extending inwardly towards a distal end. Near the distal end, the fingers 42 have a slight chamfer 45 with an inwardly sloping configuration. The combination of fingers 42 and slots 41, as shown, form a somewhat hemispherical bulbous surface extending toward the distal end or opening. On the exterior surface of the saddle 40 at the proximal end is shown a pair of opposed arcuate convex projections 50 extending partially toward the fingers 42. These projections 50 fix the alignment of the saddle 40 in the tulip 20. The tulip 20 has a pair of opposed arcuate concave slots 52 for receiving the projections 50. The slots 52 being closely sized to receive the projections 50 fix the saddle 40 and tulip 20 so the rod 60 receiving openings or slotted opening 22 and recess 44 are fixed in alignment.

With reference to FIG. 4, when the saddle 40 and tulip 20 assembly 10 are positioned over the head 4 of the bone screw 2, the fingers 42 are configured to have an internal surface or cavity 43 that matches or is complimentary to the surface of the head 4 of the bone screw 2. As illustrated, the head 4 of the bone screw 2 is either at least partially or fully hemispherical and the saddle 40 has the complimentary internal surface or cavity 43 adapted to receive the head 4 of this bone screw 2 in such a fashion that when the bone screw 2 is entered into the saddle 40, the fingers 42 will receive the head 4 of the bone screw 2. The fingers 42 are designed with a sufficient length, thickness and width that they will deflect or flexure outwardly or inwardly. On assembly, the fingers 42 move slightly outwardly as the chamfers 45 slide over the surface of the head 4 past the maximum diameter Dmax of the head 4 of the bone screw 2 and slide past the maximum diameter Dmax until fully seated inside the saddle 40 as illustrated in FIG. 4. When this occurs, the saddle 40 and the head 4 of the bone screw 2 are still able to move proximally or distally because the assembly 10 has not been tightened and the fingers 42 will hold the head 4 loosely so the saddle 40 can move angularly about head 4.

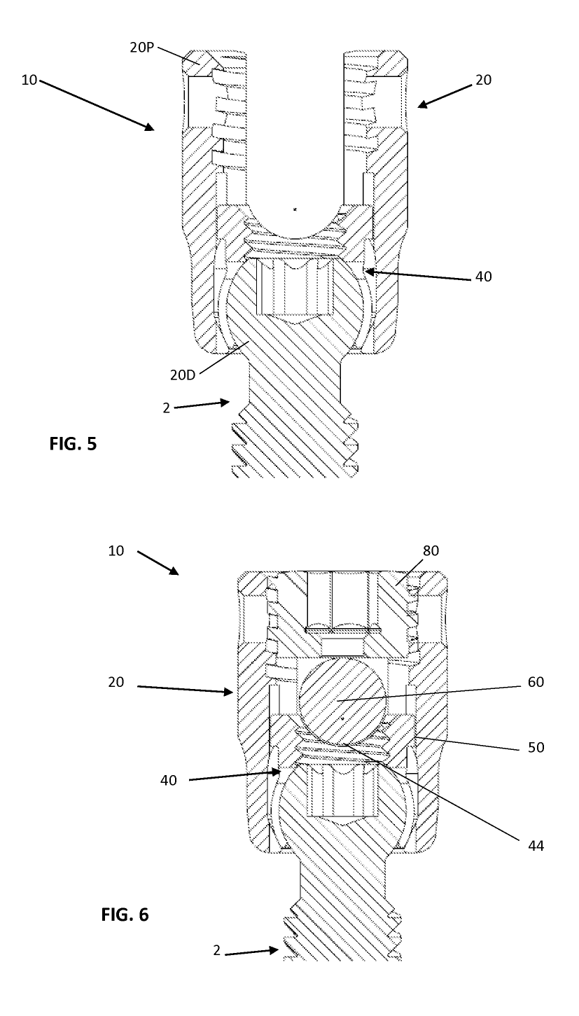

With further reference to FIG. 5, the saddle 40 when tightened, will move directionally towards a distal end 20D of the tulip 20. The distal end 20D of the tulip 20 has a conical shape in the recess such that it tapers inwardly, this conical shape is designed to be larger than the maximum diameter of the head 4 of the bone screw 2 so the head 4 of the bone screw 2 can freely pass into the tulip 20, but on entry will engage the saddle 40 adjacent the interior surface 43 of the fingers 42, as previously mentioned. As the saddle 40 is tightened, it moves toward the distal end 20D, the fingers 42 near the distal end of the saddle 40 mate with the conical surface of the tulip 20 and compress inwardly tightening the fingers 42 about the head 4 of the bone screw 2. The fingers 42 deflect inwardly compressing and tightening and due to the fact that they are below the maximum diameter of the bone screw 2, when this occurs the bone screw 2 is rigidly and securely held by the tightened saddle 40.

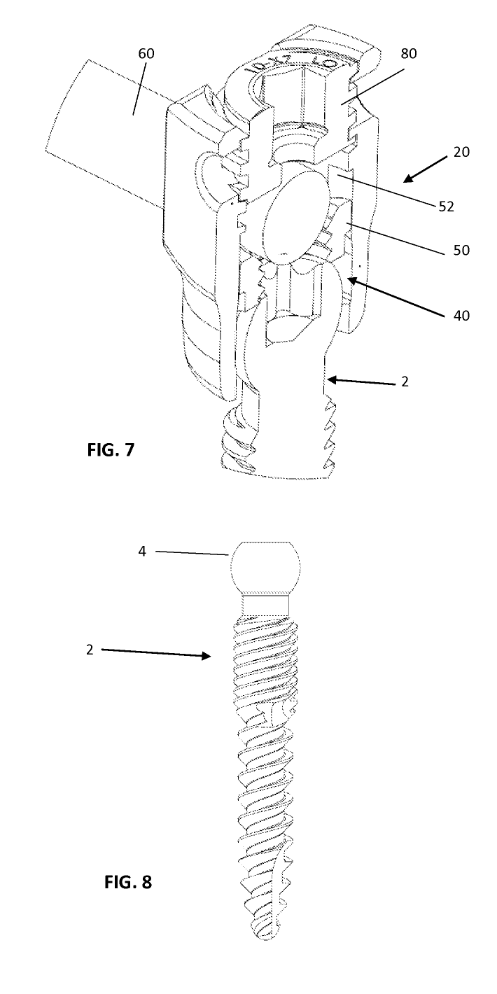

With reference to FIGS. 6 and 7, in order to accomplish this initial tightening of the tulip 20 and saddle 40 assembly 10 against the hemispherical or at least partially hemispherical head 4 of the bone screw 2, there is illustrated a rod 60 and set screw 80. The rod 60 is shown fitting into the proximal end of the saddle 40 and tulip 20 through the slotted portion 22 and resting on the concavity 44 complimentary to the rod 60 so that it is positioned in such a fashion that it is supported inside the tulip 20 by at least the saddle 40 along this concavity 44. A set screw 80 is then inserted into the assembly 10 and tightens against the rod 60 as the rod 60 and set screw 80 are driven toward the distal end 20D the saddle 40 moves distally toward the conical distal end 20D of the tulip 20 and the fingers 42 are compressed tightly against the head 4 of the bone screw 2 past the maximum diameter of the head 4 of the bone screw 2. This creates a secure assembly and fixes both the angularity of the tulip 20 relative to the bone screw 2 and fixes the tulip 20 assembly 10 firmly onto the head 4 of the bone screw 2. Upon tightening, the assembly is now complete.

As shown, the tulip 20 and saddle 40 subassembly 10 can be positioned in such a way that they fit onto a bone screw 2, which has been driven into the bone by the use of a driver tool inserted into the tool drive opening 5, that can already be pre-positioned in a pedicle or other bone structure of a patient. In this fashion, the tulip 20 and saddle 40 combined assembly 10 are configured to be moved over and above the bone screw 2 already positioned in the bone, fit onto the head 4 of the bone screw 2, adjusted for angularity due to the fact that the saddle 40 is not tightened and therefore the tulip 20 and saddle 40 subassembly 10 are free to move in any axial direction about the hemispherical or partial hemispherical portion of the head 4 of the bone screw 2. The advantage of the present invention is that the angularity and fixing assembly 10 on the bone screw 2 to the head 4 is all accomplished at one element, the saddle 40 as the rod 60 and set screw 80 are assembled and tightened. The movement of the saddle 40 having a plurality of flexible, compressible and deflectable fingers 42 having an inner surface 41 complimentary to the head 4 of the screw 2, but past the maximum diameter Dmax ensures that upon compression, the entire assembly 10 is fixed so that the tulip 20 cannot be pulled or slipped off the bone screw 2 once affixed. This creates a tremendous advantage over prior art devices requiring additional components. The present invention achieves this locking of the tulip assembly 10 to the head 4 of the bone screw 2 in a rather simple and reliable manner.

With reference to FIGS. 8-10, FIG. 8 is a view of an exemplary pedicle or bone screw 2 used with the present invention. FIG. 9 is a view of the rod 60 and FIG. 10 is a view of the set screw 80 used with the present invention.

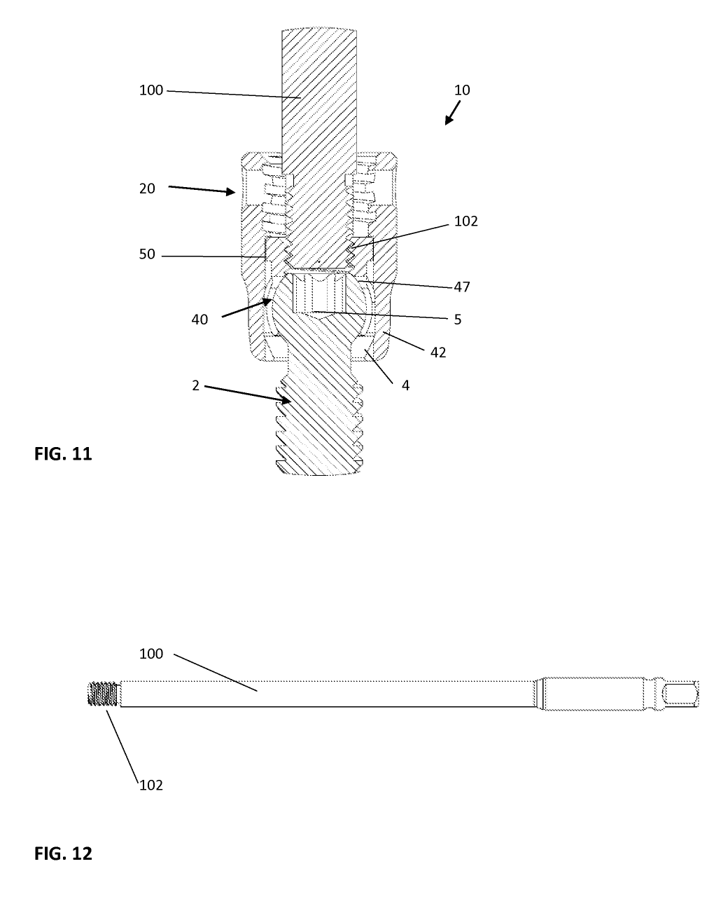

With reference to FIGS. 11 and 12, a release tool 100 can be provided. As shown in FIG. 11, when the tulip 20 and saddle 40 are snapped over the head 4 of the bone screw 2, a release tool 100 with external threads 102 can thread into the threads 47 to move back over the maximum diameter Dmax of the head 4 to allow the saddle 40 to unlock from the screw 2. Thereafter, the tulip 20 with saddle 40 can be released from the screw head 4.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims.

1. A modular head bone screw assembly comprising:
a tulip; the tulip having a slotted opening for receiving a rod, the tulip being defined by a pair of sides, one on each side of the slotted opening, the sides having a proximal end with threads for engaging a set screw to secure a rod and an annular tapered distal end with an opening for receiving and securing a bone screw, the sides have opposing concave arcuate slots positioned interposed between the threads and tapered distal end;
a saddle being internal of the tulip positioned in a recess inside the tulip, the saddle having a proximal end for engaging the rod and a distal end for receiving the bone screw, the saddle having an exterior surface positioned between the ends, the exterior surface being sized to move axially inside the tulip aligned by a pair of complimentary convex arcuate projections, each projection configured to enter one of said opposing slots and sized to move proximal in the slot to an end of the slot upon insertion of the saddle over a screw head, the saddle having a plurality of arcuate fingers positioned to create a bulbous exterior shape with an interior receiving chamber complimentarily shaped relative to a head of a bone screw, the arcuate fingers separated by slots extending from near the proximal end through the distal end in an initial pre-loaded position, the plurality of arcuate fingers collectively being larger in diameter than the opening of the annular tapered distal end of the tulip; and
wherein upon insertion of the tulip over the head of the bone screw, the saddle moves proximally over the head simultaneously causing the arcuate fingers to flex and move past a maximum diameter of the head holding the head loosely in the complimentary shaped interior receiving chamber above the distal tapered end of the tulip and upon tightening the rod by a set screw, the saddle moves distally as the plurality of arcuate fingers are compressed about the head of the bone screw thereby fixing the bone screw into the tulip by compressing the arcuate fingers against a conical shape or surface in the recess at the distal tapered end of the tulip, the conical shape having a size greater than the diameter of the head of the bone screw insuring the head of the screw freely passes into the tulip prior to engaging the fingers of the saddle past the distal tapered end.

2. The modular head bone screw assembly of claim 1 wherein the saddle has each finger having an arcuate shape with an inwardly positioned chamfered end for sliding on the surface of a head of a bone screw thereby flexing the plurality of arcuate fingers.

3. (canceled)

4. The modular head bone screw assembly of claim 1 wherein the relaxed outer diameter of the bulbous exterior of the saddle is larger than a distal opening of the tulip.

5. The modular head bone screw assembly of claim 1 further comprises a bone screw.

6. The modular head bone screw assembly of claim 5 wherein the bone screw has one of the following head shapes; at least partially a hemispherical or spherical head.

7. The modular head bone screw assembly of claim 1 wherein the saddle has at least six or more fingers separated by slots.

8. The modular head bone screw assembly of claim 7 wherein an inner surface of the saddle along an inner surface of the plurality of fingers forms the complimentary head receiving chamber in a hemispherical or at least partial hemispherical shape sized to pass over and past the maximum diameter of the head of the bone screw.

9. The modular head bone screw assembly of claim 1 wherein each finger has a length, width or thickness sufficiently compliant to flexure or deflect inward or outward to expand or contract upon assembly and tightening.

10. The modular head bone screw assembly of claim 5 wherein the head having a driving feature for torsionally driving the screw into bone.

11. The modular head bone screw assembly of claim 1 further comprises a set screw.

12. The modular head bone screw assembly of claim 1 further comprises a rod.

13. (canceled)

14. (canceled)
